(19) United States
(12) Patent Application Publication
(10) Pub. No.: US 2018/0139987 A1
(43) Pub. Date: May 24, 2018

(54) PHARMACEUTICAL POWDER COMPOSITION AND ITS USE IN THE CONTROL OF POST-PRANDIAL GLYCAEMIC PROFILE

(71) Applicant: OMNIBLEND INNOVATION PTY LTD, Campbellfield, Victoria (AU)

(72) Inventors: Gottfried LICHTI, Essendon, Victoria (AU); Christopher Walter LICHTI, Essendon, Victoria (AU)

(73) Assignee: OMNIBLEND INNOVATION PTY LTD, Campbellfield, Victoria (AU)

(21) Appl. No.: 15/568,045

(22) PCT Filed: Apr. 22, 2016
(86) PCT No.: PCT/AU2016/050292

§ 371 Date: Oct. 20, 2017

(30) Foreign Application Priority Data
Apr. 23, 2015 (AU) 2015901459

(51) Int. Cl. A23L 2/66; A23L 2/39; A23L 7/10; A61K 31/702; A61K 35/20; A61K 38/01; A61K 47/42; A61K 47/36; A61K 9/00; A61K 9/14

(52) U.S. Cl. CPC A23L 2/66; A23L 2/39; A23L 7/101; A61K 31/702; A61K 9/146; A61K 38/018; A61K 47/42; A61K 47/36; A61K 9/0095; A61K 35/20

(57) ABSTRACT

A powder composition for preparation of a drink for control of post prandial glycaemic profile, the powder composition comprising: ⋅alpha-glucosidase inhibitor selected from the group consisting of acarbose, miglitol, voglibose and mixtures thereof, in an amount in the range of from 0.01% to 15% w/w of the powder composition; ⋅viscosifying agent in an amount in the range of 5% to 30% w/w of the powder composition; protein in an amount in the range of 40% to 90% w/w of the pharmaceutical powder composition. 10

### FIELD

The invention relates to a pharmaceutical powder composition comprising an alpha-glucosidase inhibitor for preparation of a drink for oral use, to a unit dose of the powder for preparation of a drink, a process and system for control of post prandial glycaemic profile by, for example reducing or preventing post-prandial glucose spikes. This control is important in treatment and management of Type 2 diabetes, pre-diabetes also referred to as impaired glucose tolerance (IGT), insulin resistance, and related disorders.

### BACKGROUND

Hyperglycaemia, including post-prandial hyperglycaemia, is a disorder that is accompanied by, or presents risk factors for conditions such as Type H diabetes mellitus, cardiovascular disease, glucose intolerance, hyperinsulinemia.

Many pharmaceuticals used in treatment of hyperglycaemia, are associated with significant side effects, which in some cases lead to patients suffering discomfort and having to discontinue or change medication.

Alpha-glucosidase inhibitors reliably decrease the blood sugar increase after eating. Gastrointestinal symptoms, however, are the common reaction to alpha-glucosidase inhibitors such as Acarbose. In U.S. placebo-controlled trials, the incidences of abdominal pain, diarrhea, and flatulence were 19%, 31%, and 74% respectively in 1255 patients treated with Acarbose tablets 50-300 mg t.i.d., whereas the corresponding incidences were 9%, 12%, and 29% in 999 placebo-treated patients. In a one-year safety study, during which patients kept diaries of gastrointestinal symptoms, abdominal pain and diarrhea tended to return to pretreatment levels over time, and the frequency and intensity of flatulence tended to abate with time. The increased gastrointestinal tract symptoms in patients treated with alpha-glucosidase inhibitors are a manifestation of their mechanism of action and are related to the presence of undigested carbohydrate in the lower GI tract.

There is a need for corn positions that minimize or suppress the side effects caused by alpha-glucosidase inhibitors and/or which allows dosages required to achieve the required therapeutic result to be significantly reduced.

The discussion of documents, acts, materials, devices, articles and the like is included in this specification solely for the purpose of providing a context for the present invention. It is not suggested or represented that any or all of these matters formed part of the prior art base or were common general knowledge in the field relevant to the present invention as it existed before the priority date of each claim of this application.

### SUMMARY

There is provided a powder composition for preparation of a drink for control of post prandial glycaemic profile, the composition comprising:
Alpha-glucosidase inhibitor, preferably selected from the group consisting of acarbose, miglitol, voglibose and mixtures thereof, in an amount in the range of from 0.01% to 15% w/w (preferably from 0.05 to 10% w/w) based on the powder composition;
viscosifying agent, preferably a galactomannan gum, is preferably present in the range 5 to 30% w/w of the powder, and more preferably in the range 10 to 20% w/w of the powder composition;
protein in an amount is preferably in the range 40 to 90% and more preferably 50 to 80% of the pharmaceutical powder composition.

The powder composition is particularly useful in treatment of subjects suffering diabetes or pre-diabetes.

In a further aspect there is provided a powder composition in unit dose form comprising:
Alpha-glucosidase inhibitor, preferably selected from the group consisting of acarbose, miglitol, voglibose and mixtures thereof, in an amount of from 5 mg to 250 mg, preferably from 5 mg to 100 mg such as from 10 mg to 50 mg.
protein in an amount of at least 8 g per serving on a dry weight basis. The range of 8 g to 40 g is preferred particularly 8 g to 30 g, more preferably 10 g to 25 g and most preferably 15 g to 25 g per unit dose.
viscosifying agents (preferably polysaccharide, more preferably galactomannan gum, more preferably selected from guar gum and derivatives thereof) in an amount no more than 10 g per serving (such as no more than 8 g per serving or no more than 7 g per serving) and preferably at least 1 g per serving such as at least 1.5 g per serving or at least 2 g per serving and most preferably in the range of from 3 g to 6 g per unit dose.

In a particularly preferred set of embodiments there is provided a unit dose powder composition comprising:
8 g-30 g protein;
1 g-8 g soluble fibre viscosifying agent; and
5 mg-100 mg acarbose.

The preferred composition comprises from 10 mg to 50 mg of acarbose. The powder composition is preferably in the form of finely divided powder, wherein the component powders comprise acarbose, viscosifying agent, protein and any optional auxiliary agents, are in intimate admixture, preferably the particle size is less than 500 microns and more preferably the viscosifying agent, protein and pharmaceutical are of size in the range less than 100 microns, even more preferably less than 10 microns. In one preferment the pharmaceutical active in the form of a fine powder has been coated with a coating that improves taste and mouth feel.

In a further set of embodiments the powder composition is mixed with aqueous liquid in a vessel and the resulting liquid is subsequently consumed. The powder composition comprising a unit dose with the amounts of components referred to above is preferably mixed with an aqueous liquid in an amount in the range of from 20 ml to 400 ml, preferably 30 ml to 300 ml and most preferably from 75 ml to 250 ml such as 75 ml to 200 ml or 75 ml to 150 ml.

In one set of embodiments there is provided a mixture of the pharmaceutical powder composition as described above and an aqueous liquid providing a drink wherein the drink exhibits shear-banding or eccentric shear banding (preferably at least eccentric shearbanding) flow characteristics.

In one set of embodiments the invention provides an aqueous alpha-glucosidase composition formed by combining powder with an aqueous liquid to provide a drink composition comprising wherein the ratio of the weight of components of the powder composition to aqueous liquid volume is
8 g-30 g protein; 1.5 g-8 g soluble fibre viscosifying agent; and 5 mg-100 mg acarbose per 150 ml of aqueous liquid.
   More preferably the ratio of the weight of components of the powder composition to aqueous liquid volume is
8 g-30 g protein; 1.5 g-8 g soluble fibre viscosifying agent; and 10 mg-50 mg acarbose per 150 ml of aqueous liquid.

The volume of aqueous liquid is preferably 30 ml to 300 ml and more preferably from 75 ml to 200 ml. Accordingly where the volume of water is varied from 150 ml the weight of powder components is varied accordingly. For example if the volume of water is 100 ml the amount of powder components used is 100/150 fraction of the weight per 150 ml.

The invention further provides a method of controlling post-prandial glycaemic profile of a subject, preferably a subject suffering diabetes or pre-diabetes, comprising:
providing a powder composition as herein described;
combining the unit dose of powder with aqueous liquid to provide a drink composition;
   and the subject consuming the drink preferably within 30 minutes to 0 minutes (immediately before) before a meal and more preferably from 15 minutes to immediately before a meal.

In a further set of embodiments there is provided a system for use in management of diabetes and prediabetes, comprising a powder composition in a unit dose as described above and a container comprising a base, side wall and closure and having a side wall marked to indicate a level above the base corresponding with a volume in the range of from 20 ml to 400 ml, preferably 30 ml to 300 ml and most preferably from 75 ml to 250 ml such as 75 ml to 200 ml or 75 ml to 150 ml and preferably a volume above the mark providing a volume, with the closure in place of at least one quarter, preferably at least one third of the volume below the mark to allow dispersion of the pharmaceutical powder composition in aqueous liquid by shaking the container containing the powder composition and aqueous liquid.

In one set of embodiments the powder composition is for preparation of a drink by mixing the powder with an aqueous liquid and is for oral consumption within 5 minutes of mixing the powder with an aqueous liquid.

In one set of embodiments the drink is for consumption prior to or with a meal, preferably before a meal and more preferably in a period from 60 minutes before a meal (preferably from 30 minutes before a meal) up to being consumed immediately before the meal or with the meal (preferably up to 5 minutes before the meal). More preferably the drink is for consumption in the range of from 30 minutes before the meal to immediately before a meal and more preferable from 15 minutes before a meal to immediately before a meal.

In a set of embodiments, the powder composition provides a drink on mixing with aqueous liquid wherein the drink exhibits shear-banding or eccentric shear-banding as herein described.

### DETAILED DESCRIPTION

As used herein, the term post-prandial glycaemic profile refers to a graph of the blood glucose concentration versus time after consumption of a meal. Management of post-prandial glycaemic profile refers to reducing the area under the curve and/or reducing the peak in the profile which may be described as a general flattening of the profile. This general flattening of the profile is advantageous to significantly improve management of diseases such as Type II diabetes, insulin resistance and pre-diabetes or IGT. The general flattening of the profile in those at risk of developing diabetes can prevent or delay the onset of diabetes. People at risk of diabetes are generally over 40 years old such as over 50 years old. People with close relatives having type II diabetes, people with high body weight relative to the ideal and people with non-caucasian genetics.

Throughout the description and the claims of this specification the word “comprise” and variations of the word, such as “comprising” and “comprises” is not intended to exclude other additives, components, integers or steps.

The term “aqueous liquid” in relation to the drink composition refers to any suitable drink liquid such as water, fruit and vegetable juice, milk or the like. Generally water is preferred.

The term “unit dose” refers to a dose of medicine presented in a form for consumption as an individual dose of the medicine and which may be in a container such as a packet or sachet. In the present invention the unit dose is preferably in the form of a measured portion of powder which may be in a container such as a packet or sachet which can be opened to facilitate mixing of the powder with aqueous liquid to facilitate consumption of a drink of aqueous liquid in which the single dose of powder is dispersed.

We have found, in accordance with the invention that the combination of protein and viscosifying agent provides an improvement in acarbose efficacy in controlling post-prandial glycaemic profile which is a significant improvement in the control provided by acarbose and protein or acarbose and viscosifying agent. Indeed we have found that while acarbose alone is effective in controlling post-prandial glycaemic profile the combination of acarbose, protein and viscosifying agent allow a drink composition to significantly enhance the efficacy of acarbose. This improvement in efficacy is also accompanied by reduced undesirable side effects allowing the dose to be reduced to maintain efficacy compared with acarbose alone of from the dose to be maintained with the result of improved efficacy. We found that when acarbose and protein (without fibre) were combined in a drink and used under the same conditions as the method of the invention, post-prandial glycemic control was either not improved (relative to acarbose alone) or was actually made worse.

Further, while viscosifying agents such as gums have been reported to enhance glycaemic control when used alone the formulation of a drink composition is problematic due to the propensity for viscosifying agents such as guar gum which when used in effective amounts rapidly form a jel, which is unpleasant for subjects to drink. The combination of protein and viscosifying agent allows a drink to be readily formed from a powder composition which enhances, significantly the efficacy of acarbose allowing side effects to be reduced and allows a pleasant drink to be readily prepared and consumed before a meal.

Specific examples of alpha-glucosidase inhibitors include O-4,6-didesoxy-4-[(1S,4R,5S,5S)-4,5,6-trihydroxy-3-(hydroxymethyl)-2-cyclohexan-1amino]-α-D-glucopyranosyl-(1→4)-O-α-D-glucopyranosyl((1→4)-D-glucopyranose, more commonly known as acarbose;

2(S),3(R),4(S),5(S)-tetrahydroxy-N-[2-hydroxy-1-(hydroxymethyl)-ethyl]-5-(hydroxymethyl)-1(S)-cyclohexamine, also known as voglibose; and

1,5-dideoxy-1,5-[(2-hydroxyethyl)imino]-D-glucitol, also known as miglitol.

The pharmaceutically active agent acarbose is preferably used in a unit dose of the powder composition in an amount of from 5 mg to 250 mg, more preferably from 5 mg to 100 mg such as 10 mg to 50 mg.

The viscosifying agent is generally a water swellable agent or agent complex and preferably causes the viscosity of the resulting liquid to rise to at least 600 cp more preferably at least 3000 cp within 15 minutes of admixture preferably within 10 minutes of mixing. The viscosifying agent preferably comprises a polysaccharide soluble fibre or gum such as guar gum, fenugreek gum, galactomannan gum, xanthan gum, psyllium fibre/gum and other comparable agents known to the art.

The preferred viscosifying agent is a galactomannan gum, particularly preferably selected from guar gum, fenugreek and derivatives thereof. In one set of embodiments the total viscosifying agent (preferably the total galactomannan gum) content is preferably in the range 5-30% w/w of the powder composition, and more preferably in the range 10-20% w/w of the powder composition.

The viscosifying agent, preferably galactomannan gum, particularly preferably selected from guar gum and derivatives thereof is preferably present in an amount of no more than 10 g per unit dose (such as no more than 8 g per unit dose or no more than 7 g per unit dose) and preferably at least 1 g per unit dose such as at least 1.5 g per unit dose or at least 2 g per unit dose and most preferably in the range of from 3 g to 6 g per unit dose.

There is provided a drink for treatment of a subject suffering type 1 or type 2 diabetes or impaired glucose tolerance (IGT), the drink providing a unit dose of dispersed pharmaceutical powder composition and comprising:
Alpha-glucosidase (preferably selected from acarbose, miglitol, voglibose and mixtures thereof, more preferably acarbose) in an amount of 5 to 250 mg, preferably 5 mg to 100 mg and more preferably 10 mg to 50 mg.
protein in an amount of at least 8 g per serving on a dry weight basis. The range of 8 g to 40 g is preferred particularly 10 g to 35 g, more preferably 10 g to 25 g and most preferably 15 g to 25 g protein based on the weight of the powder composition.
viscosifying agents (preferably galactomannan gum, more preferably selected from guar gum and fenugreek gum and derivatives thereof) in an amount no more than 10 g per serving (such as no more than 8 g per serving or no more than 7 g per unit dose) and preferably at least 1 g per serving such as at least 1.5 g per unit dose or at least 2 g per unit dose and most preferably in the range of from 3 g to 6 g per unit dose.
aqueous liquid in a volume of from 20 ml to 400 ml, preferably 30 ml to 300 ml and most preferably from 75 to 250 ml per unit dose such as 75 ml to 200 ml or 75 ml to 150 ml per unit dose.
optionally auxiliary materials (such as sweeteners or flavouring agents) in an amount of no more than 15 g preferably no more than 10 g such as 0 g to 5 g or such as 0.5 g to 2 g.

The pharmaceutical powder composition preferably comprises a protein such as an animal or vegetable protein more preferably dairy protein such as whey protein, casein and mixtures thereof, egg white protein and soya protein. Examples of protein of plant origin include such protein from soy bean. More preferably the protein is selected from dairy protein, particularly whey protein, casein or mixtures thereof and derivatives thereof such as hydrolysed dairy whey. Most preferably the protein is whey protein concentrate, casein, natural whey protein, whey protein isolate, milk protein concentrate and mixtures of two or more thereof.

In a preferred set of embodiments the drink prepared from the pharmaceutical protein composition exhibits shear-banding or eccentric shear banding flow characteristics when measured according to the protocol provided. Shear-banding and measurement of shear-banding is described in International Patent Publication No WO2013/173874.

Eccentric shear banding is described in International application No PCT/AU2014/001064.shear-banding.

Generally it is most preferred that the drink exhibit eccentric shear banding.

Shear-banding in a liquid driven by a rotating cylinder is characterised by the existence of (1) a band or region of high shear proximal to the rotating cylinder and (2) a band region that does not exhibit significant shear. The presence of shear-banding may be recognised in many cases by the existence of a visually apparent interface between the bands of relatively high shear and band which does not exhibit significant shear.

Centric shear-banding in a liquid is determined using a drive shaft such as a rapidly rotating cylinder in the centre of a circular container, and the presence of shear-banding may be visually observed using a dye drop spaced from the drive shaft. This is described in detail in the examples section of International Application PCT/AU2013/000537.

Eccentric shear-banding, on the other hand, is determined using a drive shaft located in an eccentric position near the wall of the container as described in the Examples section of this application.

In the presence of thickeners, fibre or the like the composition may gradually increase in viscosity if formed by mixing a dry powder composition with water. In such embodiments the determination of the presence of shear-banding is determined at 10 minutes after the commencement of vigorous mixing of the dry composition with water. This applies to both centric shear-banding and eccentric shear-banding.

In some instances, liquids may be shear-banding in an eccentric shear-banding test and non-shear-banding in a centric shear-banding test.

The resultant drink prepared from the pharmaceutical powder composition is generally for consumption within 5 minutes from mixing of the pharmaceutical powder composition with an aqueous liquid, preferably within 3 minutes. The drink may be prepared by adding the powder to the aqueous liquid or by adding the aqueuous liquid to the powder composition. Generally it is preferred in order to obtain optimum performance that the powder composition is added to the aqueous liquid and vigorously mixed or shaken with the liquid prior to consumption.

Acarbose is a pharmaceutical of the class known as alpha-glucosidase inhibitors. Examples of other alpha-glucosidase include Miglitol and Voglibose. Even though the drugs have a similar mechanism of action, there are subtle differences between acarbose and miglitol. Acarbose is an oligosaccharide, whereas miglitol resembles a monosaccharide. Miglitol is fairly well absorbed by the body, as opposed to acarbose. Moreover, acarbose inhibits pancreatic alpha-amylase in addition to alpha-glucosidase.

Acarbose also blocks pancreatic alpha-amylase in addition to inhibiting membrane-bound alpha-glucosidases. Pancreatic alpha-amylase hydrolyzes complex starches to oligosaccharides in the lumen of the small intestine.

Since alpha-glucosidase inhibitors prevent the degradation of complex carbohydrates into glucose, the carbohydrates will remain in the intestine. In the colon, bacteria will digest the complex carbohydrates, thereby causing gastrointestinal side effects such as flatulence and diarrhea. Since these effects are dose-related, it is generally advised to start with a low dose and gradually increase the dose to the desired amount. Pneumatosis cystoides intestinalis is another reported side effect. If a patient using an alpha-glucosidase inhibitor suffers from an episode of hypoglycemia (e.g. as a result of taking insulin), the patient should eat something containing monosaccharides, such as glucose tablets. Since the alpha-glucosidase inhibitor drug will prevent the digestion of polysaccharides (or non-monosaccharides), non-monosaccharide foods may not effectively reverse a hypoglycemic episode in a patient taking an alpha-glucosidase inhibitor. In general we have found that the pharmaceutical powder composition taken as a drink by mixing with an aqueous liquid as herein described provides significantly enhanced activity (of the alpha-glucosidase inhibitor) allowing the unit dose and side effects to be significantly reduced.

Optional auxiliary agents such as fillers, and other excipients may be used in the preferred embodiments of the powder composition. Examples of fillers and other excipients are described in Handbook of Pharmaceutical Excipients (J. C. Boylan et al. eds., 1986) and in H. A. Lieberman et al., Pharmaceutical Dosage Forms: Tablets (2d ed. 1990). Excipients generally may include: disintegrants, wetting agents and adsorbents, flow improvers, diluents, and colorants, sweeteners, and flavoring agents. Preferred fillers include calcium salts and simple sugars, for example, calcium phosphates, calcium sulfates, lactose, and mixtures thereof. More preferred fillers include dicalcium phosphate, tribasic calcium phosphate, directly compressible calcium sulfate, anhydrous lactose, flowable lactose and mixtures thereof. The total of excipients is generally in the range of from 0% to 40% w/w of the pharmaceutical powder composition preferably 0% to 20% w/w.

In a preferred set of embodiments the auxiliary material comprises a flow improver. Unit doses of the powder if packaged in sachets, particularly for extended periods may become more difficult to disperse in aqueous liquids to form a drink composition. Flow improvers allow the shelf life of compositions to be extended. Preferably the flow improver is present in an amount of up to 2% w/w of the powder composition, more preferably from 0.5% w/w to 1.5% w/w. The preferred flow improver is a silica flow improver and may be a silica flow improver derived from rice. The most preferred auxiliary material for use in the powder composition is a silica flow improver and present in an amount in the range of from 0.5% w/w to 2% w/w of the powder composition.

In the most preferred set of embodiments, the powder composition is used to prepare a drink for use in moderation of post-prandial glycaemia by administration of the drink in the period 30 minutes up to consumption of the meal.

Further examples of diluents include methylcellulose and edible calcium salts, such as dicalcium phosphate, dihydrate.

The invention will now be described with reference to the following examples. It is to be understood that the examples are provided by way of illustration of the invention and that they are in no way limiting to the scope of the invention.

### EXAMPLES

### Brief Description of Drawings

In the drawings:

FIG. 1 is a schematic view an apparatus used to measure eccentric shear-banding in accordance with the invention showing the rotating spindle and liquid sample.

FIG. 2 is a view from above of a liquid sample prior to measurement of eccentric shear-banding with a dye mark placed 20 mm from the container wall.

FIG. 3 is a schematic view of the apparatus of FIG. 1 during measurement of eccentric shear-banding (when the rotating spindle is lowered into the liquid sample).

FIG. 4 is schematic view from above of a measurement of angle A° referred to in the Quantitative definition of Eccentric shear-banding.

FIG. 5 is a schematic view an apparatus used to measure centric-shear banding in accordance with the invention showing the rotating spindle and liquid sample.

FIG. 6 is a view from above of a liquid sample prior to measurement of shear banding with dye marks placed adjacent the container wall and 20 mm from the container wall.

FIG. 7 is a schematic view of the apparatus of FIG. 5 during measurement of centric-shear banding when the spindle has been lowered into the test liquid.

FIG. 8 is a view from above of a drink sample showing the result of the shear banding test identifying angle “A” (25°) subtended at the centre of the circular container by the front and rear edges of the inner dye drop.

FIG. 9 is a perspective view of an apparatus of FIG. 5 during measurement of centric-shear banding showing an inner annular region of high shear relatively rapidly flowing liquid, a torroidal region (outboard of the annular region) in which the shear and flow is significantly reduced and the interface between the two regions.

FIG. 10 is a graph comparing the variation in glycaemic profile (mM blood glucose) with time (minutes) comparing a first glycaemic test profile of a drink and powder composition in accordance with the invention with a control in accordance with Example B2.

FIG. 11 is a graph comparing the variation in glycaemic profile (mM blood glucose) with time (minutes) comparing a second glycaemic test profile of a drink and powder composition in accordance with the invention with a control in accordance with Example B2.

FIG. 12 is a graph comparing the variation in glycaemic profile (mM blood glucose) with time (minutes) comparing a third glycaemic test profile of a drink and powder composition in accordance with the invention with a control in accordance with Example B2.

FIG. 13 is a graph comparing the variation in glycaemic profile (mM blood glucose) with time (minutes) comparing a second glycaemic test profile of a drink and powder composition in accordance with the invention with a control in accordance with Example B3.

FIG. 14 is a graph comparing the variation in glycaemic profile (mM blood glucose) with time (minutes) comparing a third glycaemic test profile of a drink and powder composition not in accordance with the invention with a control in accordance with Example B3.

FIG. 15 is a graph comparing the variation in glycaemic profile (mM blood glucose) with time (minutes) comparing a first glycaemic test profile of a drink and powder composition not in accordance with the invention with a control in accordance with Example B4.

FIG. 16 is a graph comparing the variation in glycaemic profile (mM blood glucose) with time (minutes) comparing a second glycaemic test profile of a drink and powder composition not in accordance with the invention with a control in accordance with Example B4.

FIG. 17 is a graph comparing the variation in glycaemic profile (mM blood glucose) with time (minutes) comparing a first glycaemic test profile of a drink and powder composition not in accordance with the invention with a control in accordance with Example B5.

FIG. 18 is a graph comparing the variation in glycaemic profile (mM blood glucose) with time (minutes) comparing a second glycaemic test profile of a drink and powder composition in accordance with the invention with a control in accordance with Example B5.

FIG. 19 is a graph comparing the variation in glycaemic profile (mM blood glucose) with time (minutes) comparing a first glycaemic test profile of a drink and powder composition in accordance with the invention with a control in accordance with Example B6.

FIG. 20 is a graph comparing the variation in glycaemic profile (mM blood glucose) with time (minutes) comparing a first glycaemic test profile of a drink and powder composition in accordance with the invention with a control in accordance with Example B7.

### ECCENTRIC SHEAR-BANDING PROTOCOL: OBJECTIVE MEASUREMENT OF ECCENTRIC SHEAR-BANDING IN A DRINK

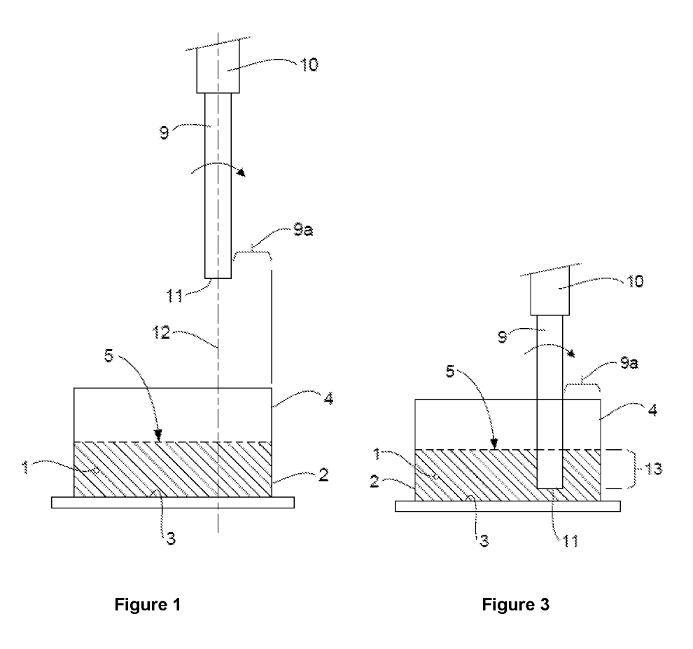

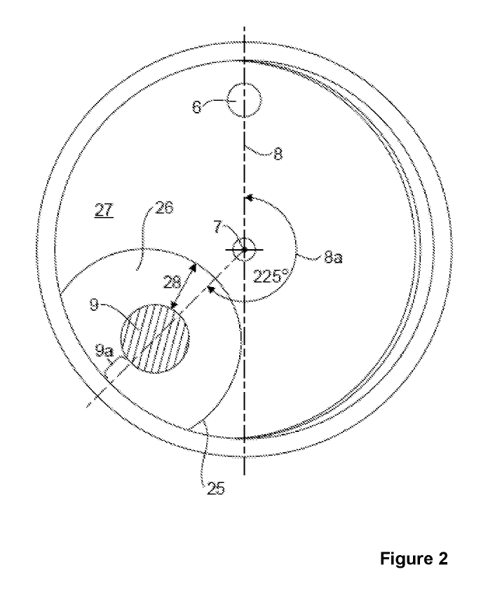

Referring to FIGS. 1 to 4 a quantity of test drink (1) containing 150 mls of water (e.g. 175 total drink weight g) is well stirred and poured into a circular flat-bottomed container (2) with a base (3) and cylindrical wall (4). The container has a diameter of 90 mm and a wall (4) height of 50 mm. The height of the surface (5) drink (1) in the container (2) is 25 mm. A drop of dye (6) is placed on a reference radius (8) at a point 20 mm in from the wall (4) of the container on a notional line on the surface of the drink through the centre (7) of the circular container (4). This drop of dye (6) will be used to define angle A as described below to determine whether eccentric shear-banding is exhibited by the sample. A smooth wooden cylinder (9) of diameter 12 mm is mounted in a rotatable chuck (10) with the axis of the cylinder (9) vertical, and the flat base (11) of the cylinder (9) is located above the drink surface. The cylinder (9) is rotated at 850 rpm.

The driven-flow aspect of the measurement is initiated by lowering the rotating cylinder (9) into the drink at a distance (9a) 15 mm from the cylindrical wall (4) of the container (2) and at an angle about the centre of the container of 225° (8a) from the reference radius (8) and position of the dye marker (6). The bottom of the cylinder (11) is lowered to a depth (13) of 20 mm below the drink surface (5). After 90 seconds, the rotation of the cylinder (9) is arrested, and the cylinder (9) is slowly withdrawn from the drink.

Quantitative Definition of Eccentric Shear-Banding in Terms of Angle A

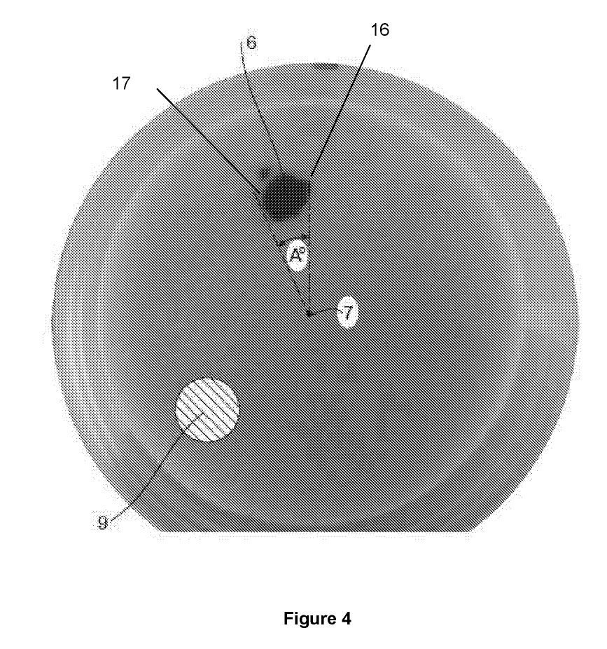

After driving the drink (1) in the container (2) by lowering the rotating cylinder (9) for 90 seconds the dye droplet (6) is inspected. The resulting droplet may be highly elongated with a front edge and a trailing edge in which the leading edge of inner dye mark (6) has become highly elongated extending through multiple revolutions about the centre (7). Alternatively the droplet may have relatively minor elongation (so that the angle subtended at the centre of the circular container is small). The angle subtended at the centre of the circular container by the front (16) and a rear edge (17) of the drop is designated angle A (see FIG. 4). If angle A is less than 40° then the liquid is considered to exhibit shear-banding behaviour. The angle A (see FIG. 4) may be measured by protractor or other suitable angle measurement apparatus.

Protocol for Determination of Eccentric Shear-Banding Interface Distances

The eccentric shear-banding test provides an annular band region of flow driven by the eccentric rotating cylinder. Compositions of the invention when subject to the above described eccentric shear-banding test, exhibit distinct bands or regions including an inner band or region about the rotating cylinder of relatively high shear (26) and rapid flow and an outer band (27) which does not exhibit significant shear and which is substantially static when compared with the inner high shear rapid flow region adjacent the rotating cylinder. In compositions of the invention the outer band or torroid region which does not exhibit significant shear and which is substantially static will include the dye drop and produce the eccentric shear-banding result as hereinbefore defined (that Angle A is less than 40°).

The interface between the two regions (25) can be readily determined by visual inspection while the cylinder is being driven during the test period. The distance (28) of the interface (25) from the rotating cylinder (9) can also be determined during this period using a ruler.

Eccentric Shear-Banding Testing of Drinks Prepared from Mixing a Powder with Aqueous Liquid

Many drinks made from reconstituted drink powder have time-variant flow characteristics. For such drinks, the following standard time sequence should be used to implement the above process. Step 1—reconstitute the drink in 150 mls of water and allow the reconstituted drink to rest for 7 minutes. Step 2—stir the rested drink and pour the drink into the above-described circular flat-bottomed container (2). After 2 minutes apply the dye drops (6) described above to the surface (5) of the drink (1), and lower the rotating cylinder (9) into the drink (1) approx. 15 mm from the container wall as described above.

The above protocol always leads to the formation of a layer of liquid that manifests local shear immediately proximal to the surface of the rotating cylinder.

In many driven drinks the shearing layer grows radially outwards from the surface of the rotating cylinder and extends throughout the liquid (although the tangential velocity of the driven drink will be significantly slower at positions further from the rotating cylinder and closer to the wall of the container). However, in drinks that exhibit eccentric shear band formation (i.e. drinks according to the current invention), a locally static layer of significant thickness (e.g. 3-20 mm or even more measured from the cylinder toward the centre of the container) develops further out from the cylinder, and this locally static outer layer coexists with the shearing inner layer. The term locally static layer means no shear or comparatively very little shear is exhibited within said layer. The simultaneous existence of an extensive shearing band and an extensive locally static band in a steady-state driven flow scenario is the characteristic feature of eccentric shear band formation.

In more general terms, eccentric shear band formation occurs in a driven-flow scenario when there is co-existence of (a) an extensive region of drink material that exhibits no local shear, and (b) an extensive region of drink material that exhibits significant local shear.

The above protocol provides a very sensitive test of eccentric\ shear band formation because an extensive shearing/rotating band is always found near the surface of the rotating cylinder, and because the shape of the red dye drop is very sensitive to the existence of local shear. Eccentric shear band formation can be detected in the above protocol whenever the liquid dye drop substantially maintains its starting shape (generally circular). In the presence of even small amounts of local shear, the liquid dye drop becomes significantly elongated in response to the local shear. This liquid-drop test for local shear is significantly more sensitive than can be achieved by introducing high-contrast solid particles to the drink (as flow markers)—this is because a solid marker will move according to the resultant forces on the solid particle, and local shear can be inferred only by comparing one particle of solid marker with a separate particle of marker.

Shear Banding Protocol: Objective Measurement of Centric Shear Banding in a Drink

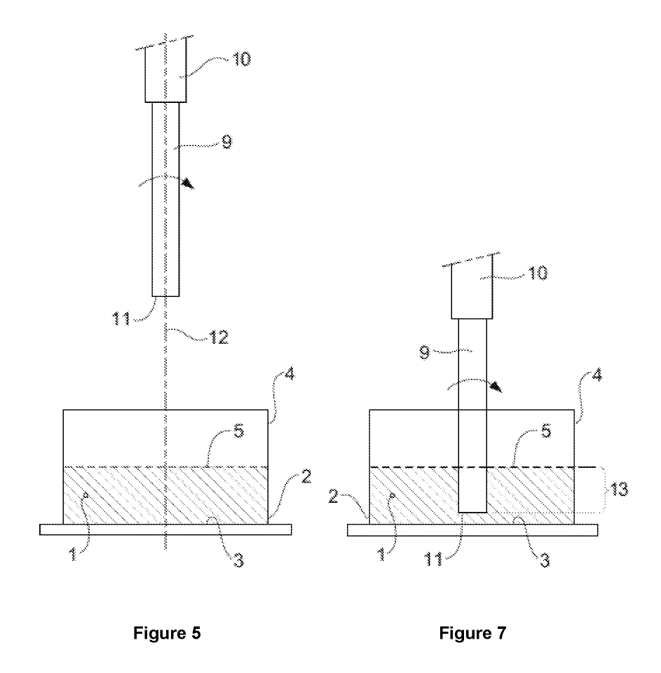

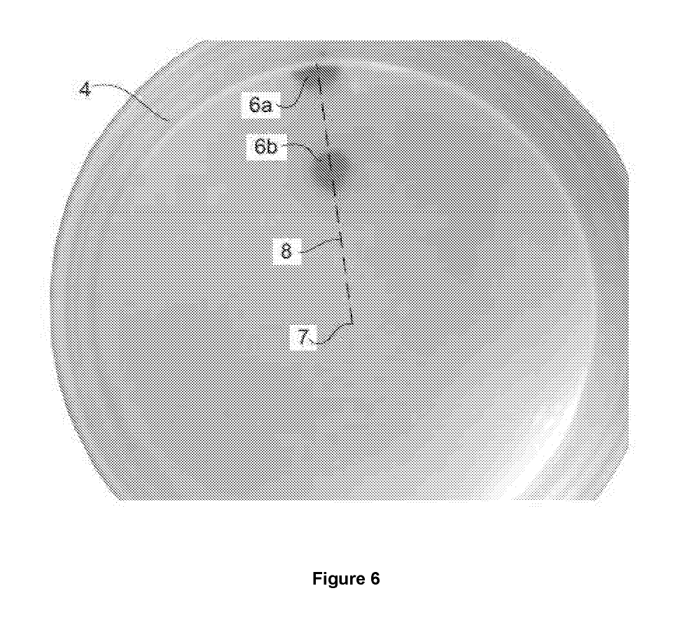

Referring to FIGS. 5 to 9 a quantity of test drink (1) containing 150 mls of water (e.g. 175 total drink weight g) is well stirred and poured into a circular flat-bottomed container (2) with a base (3) and cylindrical wall (4). The container has a diameter of 90 mm and a wall (4) height of 50 mm. The height of the surface (5) drink (1) in the container (2) is 25 mm. A drop of water-soluble dye (6a) is placed on the surface of the drink (5) close to the wall of the container (4) and a notional line on the surface of the drink from the centre (7) of the circular container and this mark (6a) is chosen as a reference diameter (8). Another drop of dye (6b) is placed on the reference diameter (8) at a point 20 mm in from the wall (4) of the container. This drop of dye (6b) will be used to define angle A as described below to determine whether shear banding is exhibited by the sample. A smooth wooden cylinder (9) of diameter 12 mm is mounted in a rotatable chuck (10) with the axis of the cylinder (9) vertical, and the flat base of the cylinder (9) is located above the drink surface in such a way that the (vertical) axis (12) of the cylinder (9) is coincident with the (vertical) axis (12) of the circular flat-bottomed container (2). The cylinder (9) is rotated at 850 rpm.

The driven-flow aspect of the measurement is initiated by lowering the rotating cylinder (9) into the drink to a depth (13) of 20 mm below the drink surface (5). After 90 seconds, the rotation of the cylinder (9) is arrested, and the cylinder (9) is slowly withdrawn from the drink.

Quantitative Definition of Shear Banding in Terms of Angle A

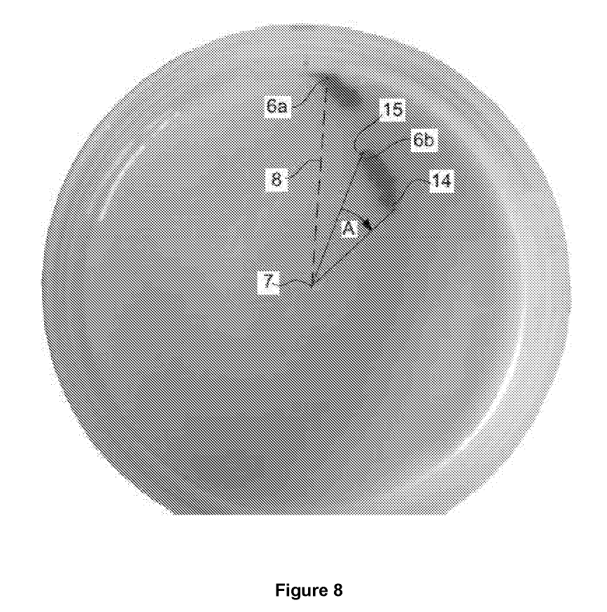

After driving the drink (1) in the container (2) by lowering the rotating cylinder (9) for 90 seconds the inner dye droplet (6b) is inspected. The resulting droplet may be highly elongated with a front edge and a trailing edge becoming highly elongated and extending through multiple revolutions about the centre as is evident from the band width of dye. Alternatively the droplet may have relatively minor elongation (so that the angle subtended at the centre of the circular container is small (see FIG. 8). The angle (A) subtended at the centre of the circular container by the front (14) and a rear edge (15) of the drop is designated angle A (see FIG. 8). If angle A is less than 40° C. then the liquid is considered to exhibit shear-banding behaviour. The angle A (see FIG. 8) may be measured by protractor or other suitable angle measurement apparatus.

Protocol for Determination of Shear Banding Interface Distances

The shear banding test provides an annular band region of flow driven by the central rotating cylinder. Compositions of the invention when subject to the above described shear banding test, exhibit distinct band or regions including an inner band or region about the rotating cylinder of relatively high shear and rapid flow and an outer band or torroid region adjacent the wall of the container in which the shear and flow is significantly reduced when compared with the inner high shear rapid flow region adjacent the rotating cylinder. In compositions of the invention the outer band or torroid region of relatively low shear and reduced flow will include the dye drop and produce the shear banding result as hereinbefore defined.

The interface between the two regions can be readily determined by visual inspection while conducting the test and the distance of the interface from the rotating cylinder determined.

The interface between inner band or region of relatively rapid flow and the outer band or torroid region of low shear and reduced flow will be described with reference to FIG. 5.

Referring to FIG. 9 the interface (25) between inner rapid flow region (26) adjacent the rotating cylinder (9) and the outer torroid region (which has a shear flow sufficiently low to produce a shear banding result) is visually evident to the naked eye and the distance (28) of the interface (25) from the rotating cylinder may be measured using a ruler placed adjacent the side of the rotating cylinder (9).

We have found that compositions which are most efficacious in moderating blood glucose levels have an annular interface spaced from the rotating cylinder by at least 2.5 mm, preferably at least 5 mm, more preferably at least 7 mm, such as at least 10 mm or at least 12 mm.

The interface will be at least 10 mm inside of the diameter at which the dye drop is placed (20 mm in from the wall). The interface is preferably no more than 18 mm from the rotating cylinder and more preferably no more than 16 mm. Accordingly, the interface will typically fall in a distance of from 2.5 mm to 18 mm from the rotating cylinder, more preferably 5 mm to 16 mm, still more preferably 7 mm to 16 mm such as 70 mm to 16 mm or from 12 mm to 16 mm.

Shear Banding Testing of Drinks Prepared from Mixing a Powder with Aqueous Liquid

Many drinks made from reconstituted drink powder have time-variant flow characteristics. For such drinks, the following standard time sequence should be used to implement the above process. Step 1—reconstitute the drink in 150 mls of water and allow the reconstituted drink to rest for 7 minutes. Step 2—stir the rested drink and pour the drink into the above-descried circular flat-bottomed container (2). After 2 minutes apply the dye drops (6a, 6b) described above to the surface (5) of the drink (1), and lower the rotating cylinder (9) into the drink (1).

The above protocol always leads to the formation of a layer of liquid that manifests local shear immediately proximal to the surface of the rotating cylinder.

In many driven drinks the shearing layer grows radially outwards from the surface of the rotating cylinder and extends throughout the liquid (although the tangential velocity of the driven drink will be significantly slower at positions further from the rotating cylinder and closer to the wall of the container). However, in drinks that exhibit shear band formation (i.e. drinks according to the current invention), a locally static layer (adjacent the wall) of significant thickness (e.g. 15-20 mm or even more) develops further out from the cylinder, and this locally static outer layer coexists with the shearing inner layer. The term locally static layer means no shear is exhibited within said layer. The simultaneous existence of an extensive shearing band and an extensive locally static band in a steady-state driven flow scenario is the characteristic feature of shear band formation.

In more general terms, shear band formation occurs in a driven-flow scenario when there is co-existence of (a) an extensive region of drink material that exhibits no local shear, and (b) an extensive region of drink material that exhibits significant local shear.

The above protocol provides a very sensitive test of shear band formation because an extensive shearing/rotating band is always found near the surface of the rotating cylinder, and because the shape of the red dye drop is very sensitive to the existence of local shear. Shear band formation can be detected in the above protocol whenever the liquid dye drop substantially maintains its starting shape (generally circular). In the presence of even small amounts of local shear, the liquid dye drop becomes significantly elongated in response to the local shear. This liquid-drop test for local shear is significantly more sensitive than can be achieved by introducing high-contrast solid particles to the drink (as flow markers)—this is because a solid marker will move according to the resultant of all forces on the solid, and local shear can be inferred only by comparing one particle of solid marker with a separate particle of marker.

### Example A1

Single-Serve (Unit Dose) Powder Sample with 25 mg Acarbose (this Sample is Prepared According to the Method of the Invention).

25 mg acarbose (half a 50 mg acarbose pharmaceutical tablet) was placed into a mortar and ground to a fine powder with a pestle. 1 g whey powder was added to the mortar with further grinding to provide a finely ground powder composite of acarbose and whey powder. This powder composite was quantitatively added to a larger powder composition comprising: (i) 20 g whey powder concentrate, and (ii) 5 g guar gum powder. The total sample was added to a plastic bag, a knot was tied at the top of the bag, and further mixing took place by shaking the bag and its contents for 2 minutes. A pre-meal drink based on this powder sample was made and used by (i) adding 150 mls water to a graduated shaking vessel, (ii) quantitatively adding the single-serve powder sample described above, (iii) capping the shaking vessel with a screw cap containing a stopper, and shaking vigorously for 5 seconds, then (iv) removing the stopper and quickly consuming the entire liquid contents of the shaker vessel.

### Example A1a

Single-Serve Powder Sample (No Acarbose) (this Sample is for a Pre-Meal Drink but Lacks Therapeutic Agent—it is not Prepared According to the Method of the Invention)

This powder sample comprised: (i) 20 g whey powder concentrate, and (ii) 5 g guar gum powder. The total sample was added to a plastic bag, a knot was tied at the top of the bag, and further mixing took place by shaking the bag and its contents for 2 minutes. A pre-meal drink based on this powder sample was made using the procedure described in example A1 above.

### Example A1b

Single-Serve Powder Sample with 12.5 mg Acarbose (this Sample is Prepared According to the Method of the Invention).

12.5 mg acarbose (a quarter of a 50 mg acarbose pharmaceutical tablet) was placed into a mortar and ground to a fine powder with a pestle. 1 g whey powder was added to the mortar with further grinding to provide a finely ground powder composite of acarbose and whey powder. This powder composite was quantitatively added to a larger powder composition comprising: (i) 20 g whey powder concentrate, and (ii) 5 g guar gum powder. The total sample was added to a plastic bag, a knot was tied at the top of the bag, and further mixing took place by shaking the bag and its contents for 2 minutes. A pre-meal drink based on this powder sample was made and used by (i) adding 150 mls water to a graduated shaking vessel, (ii) quantitatively adding the single-serve powder sample described above, (iii) capping the shaking vessel with a screw cap containing a stopper, and shaking vigorously for 5 seconds, then (iv) removing the stopper and quickly consuming the entire liquid contents of the shaker vessel.

### Example A1c

Single-Serve Powder Sample with 25 mg Acarbose (this Sample is Prepared According to the Method of the Invention).

25 mg acarbose (a half of a 50 mg acarbose pharmaceutical tablet) was placed into a mortar and ground to a fine powder with a pestle. 1 g whey powder was added to the mortar with further grinding to provide a finely ground powder composite of acarbose and whey powder. This powder composite was quantitatively added to a larger powder composition comprising: (i) 20 g whey powder concentrate, and (ii) 5 g psyllium husk. The total sample was added to a plastic bag, a knot was tied at the top of the bag, and further mixing took place by shaking the bag and its contents for 2 minutes. A pre-meal drink based on this powder sample was made and used by (i) adding 150 mls water to a graduated shaking vessel, (ii) quantitatively adding the single-serve powder sample described above, (iii) capping the shaking vessel with a screw cap containing a stopper, and shaking vigorously for 5 seconds, then (iv) removing the stopper and quickly consuming the entire liquid contents of the shaker vessel.

### Example A1d

Single-Serve Powder Sample with 25 mg Acarbose.

25 mg acarbose (a half of a 50 mg acarbose pharmaceutical tablet) was placed into a mortar and ground to a fine powder with a pestle. 1 g whey powder was added to the mortar with further grinding to provide a finely ground powder composite of acarbose and whey powder. This powder composite was quantitatively added to a larger powder composition comprising: (i) 25 g whey protein concentrate. The total sample was added to a plastic bag, a knot was tied at the top of the bag, and further mixing took place by shaking the bag and its contents for 2 minutes. A pre-meal drink based on this powder sample was made and used by (i) adding 150 mls water to a graduated shaking vessel, (ii) quantitatively adding the single-serve powder sample described above, (iii) capping the shaking vessel with a screw cap containing a stopper, and shaking vigorously for 5 seconds, then (iv) removing the stopper and quickly consuming the entire liquid contents of the shaker vessel.

### Example A1e

Single-Serve Powder Sample with 25 mg Acarbose.

25 mg acarbose (a half of a 50 mg acarbose pharmaceutical tablet) was placed into a mortar and ground to a fine powder with a pestle. 1 g whey powder was added to the mortar with further grinding to provide a finely ground powder composite of acarbose and whey powder. This powder composite was quantitatively added to a larger powder composition comprising: (i) 12.5 g milk protein isolate powder and (ii) 10.17 g whey protein concentrate powder and (iii) 0.325 g guar gum and (iv) 0.125 g xanthan gum. The total sample was added to a plastic bag, a knot was tied at the top of the bag, and further mixing took place by shaking the bag and its contents for 2 minutes. A pre-meal drink based on this powder sample was made and used by (i) adding 150 mls water to a graduated shaking vessel, (ii) quantitatively adding the single-serve powder sample described above, (iii) capping the shaking vessel with a screw cap containing a stopper, and shaking vigorously for 5 seconds, then (iv) removing the stopper and quickly consuming the entire liquid contents of the shaker vessel.

### Example A2

Standard White Rice Meal

160 g microwaveable white rice (details, amount of available carbohydrate?) was eaten together with a cup of water.

### Example A3

Standard White Rice Meal No. 2

125 g microwaveable white rice (details, amount of available carbohydrate?) was eaten together with a cup of water.

### Example A3a

Standard white rice meal No. 3

240 g microwaveable white rice (Uncle Bens Brand Microwavable White Rice, 182.4 g of available carbohydrate) and 200 ml of hot tea.

### Example A4

Standard White Bread Meal

2 slices of thick-cut savoury white bread spread with 29 g jam (over both slices) were eaten together with a cup of tea or coffee. A total available carbohydrate per serve was 50 g.

### Example A4a

Standard White Bread Meal No. 2,

2 slices of thick-cut savoury white bread spread with 29 g jam (over both slices) were eaten together with a cup of tea or coffee. 25 mg acarbose consumed with first mouthful of meal. A total available carbohydrate per serve was 50 g.

### Example A5

Protocol to Measure Post-Prandial Glycemic Profile

Finger-prick blood sugar readings were taken before consumption of the standard meal in the morning (i.e. the standard meal was breakfast). Two readings were taken and the average value was used as the blood sugar reading. After consumption of the meal, another blood sugar reading was taken. Further blood sugar readings were taken at 15-minute intervals thereafter.

### Example B1

A subject (HP2, Healthy Participant, Male, Age 30,) was given a standard white rice breakfast, containing 125 g cooked white rice (example A3), and was tested for post-prandial glycemic profile—this was the first control glycemic profile. On another day, the same subject was given a tablet containing 25 mg of acarbose with the first bite of the standard white rice breakfast—this was the second control glycemic profile. On another day, the same subject was given the pre-meal drink, which did not contain acarbose (see example A1a above—this pre-meal drink was consumed immediately before the meal). This was the third control glycemic profile. On another day, the same subject was given the acarbose-containing pre-meal drink described in example A1 (this drink is prepared according to the invention). This was the test glycemicsrofile.

Healthy Participant Protocol
1) 125 g of white rice
2) 25 mg of Acarbose with 125 g of white rice
3) Premeal drink immediately prior to 125 g of white rice
4) 25 mg of Acarbose mixed into premeal drink, consumed immediately prior to 125 g of white rice.

TABLE 1
AB - HP2

| | | | | Test |
|---|---|---|---|---|
| | First | Second | Third | glycemic |
| | control | control | control | profile |
| Time | glycemic | glycemic | glycemic | (mM blood |
| after | profile | profile | profile | glucose) as |
| eating | (mM blood | (mM blood | (mM blood | according to |
| (mins) | glucose) | glucose) | glucose) | the invention |
| −15 | 5.05 | 5.65 | 5.53 | 5.6 |
| 0 | 5.83 | 6.03 | 5.75 | 5.7 |
| 15 | 7.5 | 6.4 | 6.3 | 6 |
| 30 | 8.03 | 7.04 | 7.2 | 5.75 |
| 45 | 7.35 | 6.75 | 6.3 | 5.95 |
| 60 | 7 | 5.9 | 5.75 | 6.25 |
| 75 | 6.65 | 6.1 | 5.55 | 5.75 |
| 90 | 5.9 | 6.175 | 6.05 | 5.75 |
| 105 | 6.15 | 5.8 | 6.46 | 5.83 |
| 120 | 6.25 | 6.3 | 5.8 | 6.05 |
| 135 | 5.9 | 6.23 | | 5.55 |
| 150 | 5.83 | | | 6 |
| 165 | | | | |

A subject with type 2 diabetes (T2D1, Female, Age 62,) was given a standard white rice breakfast (Example A2) and was tested for post-prandial glycemic profile—this was the first control glycemic profile. On another day, the same subject was given a tablet containing 25 mg of acarbose with the first bite of the standard white rice breakfast—this was the second control glycemic profile. On another day, the same subject was given the pre-meal drink which did not contain acarbose (see example A1a above—this premeal drink was consumed immediately before the meal). This was the third control glycemic profile. On another day, the same subject was given the acarbose-containing pre-meal drink described in example A1 (this drink is prepared according to the invention). This was the test glycemic profile.

TABLE 2
AC T2D1

| | | | | Test |
|---|---|---|---|---|
| | First | Second | Third | glycemic |
| | control | control | control | profile |
| Time | glycemic | glycemic | glycemic | (mM blood |
| after | profile | profile | profile | glucose) as |
| eating | (mM blood | (mM blood | (mM blood | according to |
| (mins) | glucose) | glucose) | glucose) | the invention |
| −15 | 9.26 | 8.8 | 9.35 | 8.9 |
| 0 | 11.1 | 8.9 | 10.2 | 8.6 |
| 15 | 11.5 | 9.13 | 10.7 | 8.75 |
| 30 | 11.4 | 9 | 10.8 | 9 |
| 45 | 10.8 | 9.6 | 10.1 | 8.52 |
| 60 | 11.2 | 9.8 | 11 | 9 |
| 75 | 11.3 | 9.6 | 11.2 | 9.42 |
| 90 | 11.1 | 10.9 | 10.5 | 8.45 |
| 105 | 11.6 | 9.65 | 10.4 | 9.06 |
| 120 | 11.2 | 10.25 | 10.6 | 9.05 |
| 135 | 10.8 | 9.82 | 9.2 | 8.4 |
| 150 | 11 | 9.26 | 8.9 | 7.6 |
| 165 | 10.9 | 9.05 | 8.7 | 7.9 |

A subject with type 2 diabetes (T2D3, Female, Age 65,) was given a standard white rice breakfast (Example A2) and was tested for post-prandial glycemic profile—this was the first control glycemic profile. On another day, the same subject was given a tablet containing 25 mg of acarbose with the first bite of the standard white rice breakfast—this was the second control glycemic profile. On another day, the same subject was given the pre-meal drink which did not contain acarbose (see example A1a above—this premeal drink was consumed immediately before the meal). This was the third control glycemic profile. On another day, the same subject was given the acarbose-containing pre-meal drink described in example A1 (this drink is prepared according to the invention). This was the test glycemic profile.

TABLE 3
AD T2D3

| | | | | Test |
|---|---|---|---|---|
| | First | Second | Third | glycemic |
| | control | control | control | profile |
| Time | glycemic | glycemic | glycemic | (mM blood |
| after | profile | profile | profile | glucose) as |
| eating | (mM blood | (mM blood | (mM blood | according to |
| (mins) | glucose) | glucose) | glucose) | the invention |
| −15 | 8.45 | | | 7.96 |
| 0 | 11.1 | | | 9.13 |
| 15 | 13.0 | | | 10 |
| 30 | 12.6 | | | 9.5 |
| 45 | 12.4 | | | 10.4 |
| 60 | 11.8 | | | 10.1 |
| 75 | 10.7 | | | 9.15 |
| 90 | 9.9 | | | 8.25 |
| 105 | 8.9 | | | 7.75 |
| 120 | 9.16 | | | 6.7 |
| 135 | 9.1 | | | 6.35 |
| 150 | 9.05 | | | 6.2 |
| 165 | 7.9 | | | 5.8 |

A subject with type 2 diabetes (T2D5, Female, Age 65,) was given a standard white rice breakfast (Example A2) and was tested for post-prandial glycemic profile—this was the first control glycemic profile. On another day, the same subject was given a tablet containing 25 mg of acarbose with the first bite of the standard white rice breakfast—this was the second control glycemic profile. On another day, the same subject was given the pre-meal drink which did not contain acarbose (see example A1a above—this premeal drink was consumed immediately before the meal). This was the third control glycemic profile. On another day, the same subject was given the acarbose-containing pre-meal drink described in example A1 (this drink is prepared according to the invention). This was the test glycemic profile.

TABLE 4
AD T2D5

| | | | | Test |
|---|---|---|---|---|
| | First | Second | Third | glycemic |
| | control | control | control | profile |
| Time | glycemic | glycemic | glycemic | (mM blood |
| after | profile | profile | profile | glucose) as |
| eating | (mM blood | (mM blood | (mM blood | according to |
| (mins) | glucose) | glucose) | glucose) | the invention |
| −15 | 6.25 | | | 5.96 |
| 0 | 8 | | | 5.75 |
| 15 | 8.7 | | | 5.7 |
| 30 | 8.75 | | | 5.9 |
| 45 | 6.86 | | | 5.3 |
| 60 | 6.23 | | | 5.75 |
| 75 | 5.8 | | | 5.65 |
| 90 | 5.55 | | | 5.75 |
| 105 | 5.86 | | | 5.55 |
| 120 | 5.75 | | | 6.05 |
| 135 | 5.95 | | | 6 |
| 150 | 6.1 | | | 5.5 |
| 165 | 5.95 | | | 5.35 |

The test glycemic profile (obtained using the method of the invention) was consistently lower than any of the 3 control profiles

It was noticed that when a 50 mg tablet of acarbose was taken with, or soon before or soon after the consumption of a pre-meal drink not containing acarbose, the resultant glycemic profile was significantly higher than if said tablet was taken together with the standard meal (i.e. the profile was higher than the second control glycemic profile in table AA). So the method of the invention provides for a better post-prandial glycemic profile than if the acarbose was delivered in tablet form alongside the pre-meal drink.

### Example B2

A subject with type 2 diabetes (T2D5, Female, Age 65,) was given a standard white rice breakfast, containing 240 g cooked white rice (example A3a) and also the same subject was given a tablet containing 25 mg of acarbose with the first bite of the standard white rice breakfast and was tested for post-prandial glycemic profile—this was the glycemic control profile. On another day, the same subject was given a pre-meal drink containing 12.5 mg acarbose, 20gWPC80, 5 g guar gum and 150 ml of water (see example A1b this premeal drink was consumed immediately before the meal)—this was the first glycemic test profile. On another day, the same subject was given a pre-meal drink, which contained 25 mg acarbose, 20 g whey protein and 5 g psyllium husk (see example A1c above—this pre-meal drink was consumed immediately before the meal). This was the second glycemic test profile. On another day the same subject was given a pre-meal drink containing 25 mg acarbose, 20gWPC80, 5 g guar gum and 150 ml of water (see example A1)—This was the third glycemic test profile.

Summary of Protocols

Control—

25 mg of acarbose with 240 g of white rice

First Glycemic Test—

12.5 mg acarbose mixed with premeal (20gWPC80+5 g guar gum) drink (made according to the method of the invention) (see example A1b) immediately before 240 g of white rice

2^{nd} Glycemic Test—

25 mg of Acarbose mixed into a premeal drink (20gWPC80, 5 g Psyllium husk) (made according to the method of the invention) (see example A1c), consumed immediately prior to 240 g of white rice.

3^{rd} Glycemic Test—

25 mg of Acarbose mixed into premeal drink (20 g WPC80, 5 g guar gum) (made according to the method of the invention) (see example A1a), consumed immediately prior to 240 g of white rice.

TABLE 5
T2D5

| | | First | Second | Third |
|---|---|---|---|---|
| | Glycemic | glycemic | glycemic | glycemic |
| Time | control | test | test | test |
| after | profile | profile | profile | profile |
| eating | (mM blood | (mM blood | (mM blood | (mM blood |
| (mins) | glucose) | glucose) | glucose) | glucose) |
| −25 | 5 | 5.7 | 5 | 7.13 |
| −10 | 5.2 | 6.15 | 5.5 | 7.42 |
| 0 | 7.4 | 6.55 | 6.35 | 8.46 |
| 15 | 8.8 | 8.1 | 7.35 | 8.97 |
| 30 | 10.4 | 9.85 | 8.2 | 9.5 |
| 45 | 10.1 | 8.7 | 9.16 | 9.85 |
| 60 | 9.7 | 8.6 | 9.75 | 9.83 |
| 75 | 9.95 | 7.7 | 10.2 | 9.9 |
| 90 | 9.2 | 7.5 | 9.55 | 9.35 |
| 105 | 8.7 | 6.9 | 8.93 | 9.4 |
| 120 | 8.73 | 7.15 | 7.35 | 8.2 |
| 135 | 8.53 | 6.93 | 6.56 | 7.3 |
| 150 | 8.56 | 6.65 | 5.95 | 7.25 |
| 165 | 7.3 | 6.2 | 5.65 | 6.5 |
| 180 | 6.5 | 5.65 | 5.35 | 5.85 |

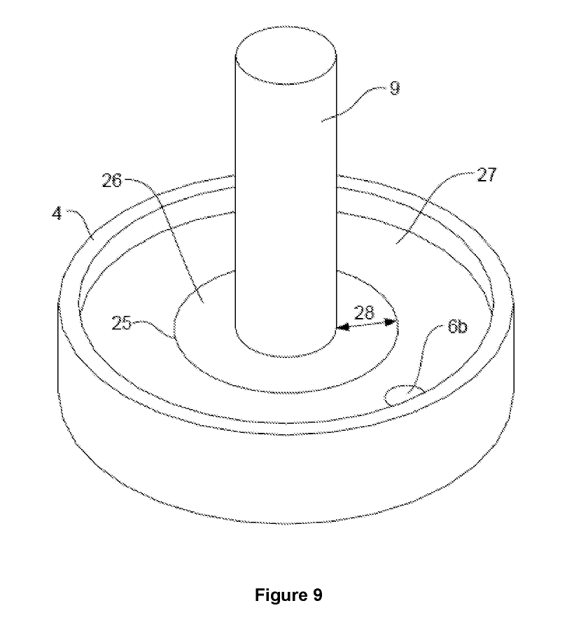

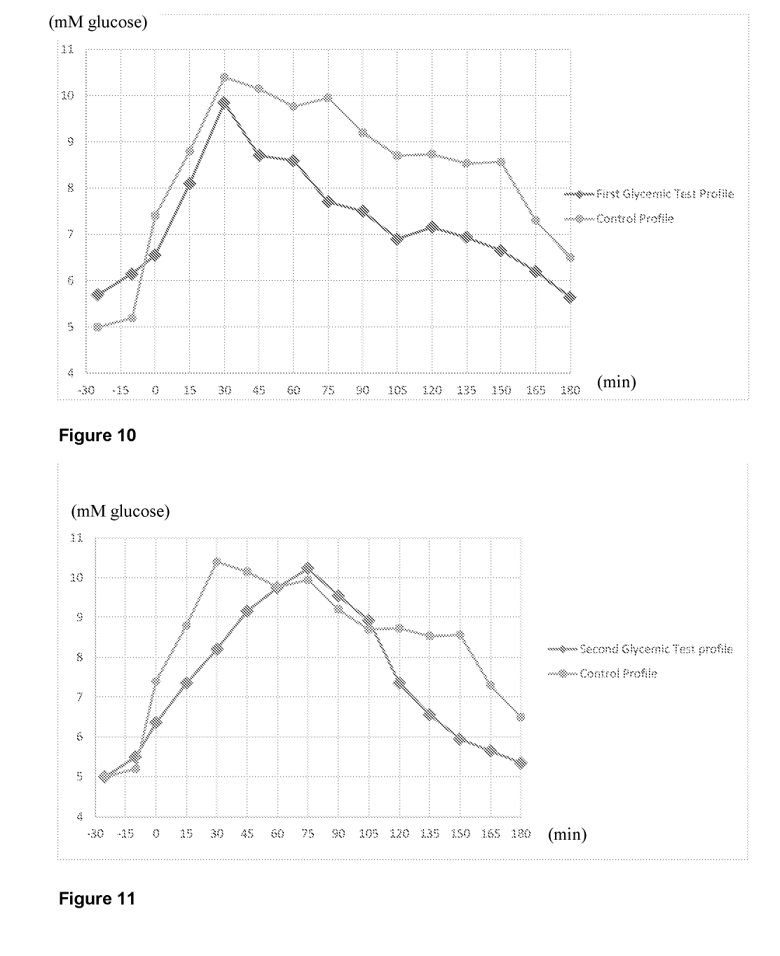

The results of the first glycaemic test profile are plotted together with the control in FIG. 10.

When participant T2D5 consumed a premeal drink containing 12.5 mg of Acarbose, 20 g whey protein and 5 g guar gum (see example A1a—made according to the method of the invention) immediately prior to 240 g of white rice:

Participant produced a better glycemic profile when compared to control—
lower post prandial glucose levels from t=30 minutes to t=180 minutes compared to control.

The results of the second glycaemic test profile are plotted againse the control profile in FIG. 11.

When participant T2D5 consumed a premeal drink containing 25 mg of Acarbose, 20 g whey protein and 5 g psyllium husk (see example A1a—made according to the method of the invention) immediately prior to 240 g of white rice:

Participant produced a better glycemic profile when compared to control—
Produced lower glucose levels from t=0 minutes to t=60 minutes and from t=120 minutes to t=180 minutes compared to control.

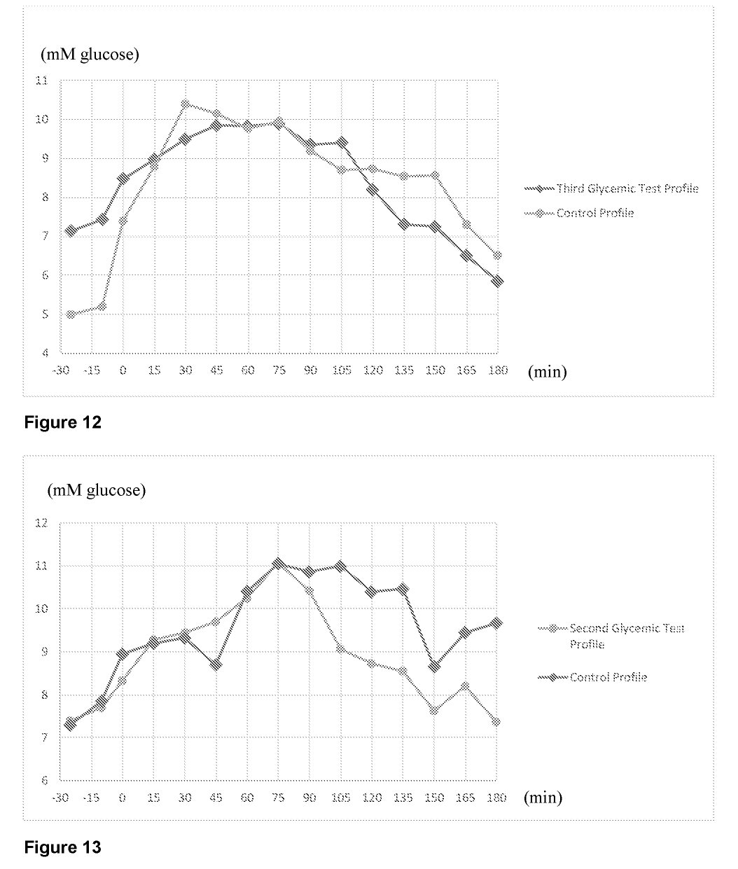

The results of the third glycaemic test profile and control profile are plotted in FIG. 12.

When participant T2D5 consumed a premeal drink containing 25 mg of Acarbose, 20 g whey protein and 5 g guar gum (see example A1a—made according to the method of the invention) immediately prior to 240 g of white rice:

Participant produced a better glycemic profile when compared to control—
Lower post prandial peak than control. If baselines of both profiles are matched, peak glucose height when taking the premeal (containing 25 mg acarbose whey protein and guar gum—made according to the method of the invention) produces a 2.85 mmol/L rise above baseline. Compared against the control showing a raise above baseline of 5.3 mmol/L.
If baselines of both profiles were matched, the glucose profile relating to the premeal containing 25 mg acarbose whey protein and guar gum (made according to the method of the invention) would be considered lower than control for the entirety of the testing time.

### Example B3

A subject with type 2 diabetes (T2D1, Female, Age 62,) was given a standard white rice breakfast, containing 240 g cooked white rice (example A3a), and also the same subject was given a tablet containing 25 mg of acarbose with the first bite of the standard white rice breakfast and was tested for post-prandial glycemic profile—this was the control glycemic profile. On another day, the same subject was given a pre-meal drink, which contained 25 mg acarbose, 20 g whey protein and 5 g psyllium husk (see example A1c above—made according to the method of the invention). This was the second test glycemic profile. On another day the same subject was given a pre-meal drink containing 25 mg acarbose, 20gWPC80, 5 g guar gum and 150 ml of water (see example A1—made according to the method of the invention)—This was the third test glycemic_profile.

Person with type 2 diabetes protocol

Control—25 mg of acarbose with 240 g of white rice

1^{st} Glycemic Test—Not Done

2^{nd} Glycemic Test—25 mg of Acarbose mixed into premeal drink (20 g WPC80, 5 g psyllium husk—made according to the method of the invention) (see example A1c), consumed immediately prior to 240 g of white rice.

3^{rd} Glycemic Test—25 mg of Acarbose mixed into premeal drink (20gWPC80, 5 g guar gum—made according to the method of the invention) (see example A1a), consumed immediately prior to 240 g of white rice.

TABLE 6
T2D1

| | | First | Second | Third |
|---|---|---|---|---|
| | | glycemic | glycemic | glycemic |
| Time | Control | test | test | test |
| after | profile | profile | profile | profile |
| eating | (mM blood | (mM blood | (mM blood | (mM blood |
| (mins) | glucose) | glucose) | glucose) | glucose) |
| −25 | 7.3 | | 7.4 | 7.55 |
| −10 | 7.85 | | 7.7 | 7.96 |
| 0 | 8.95 | | 8.33 | 9.2 |
| 15 | 9.2 | | 9.28 | 10.2 |
| 30 | 9.32 | | 9.45 | 8.85 |
| 45 | 8.7 | | 9.7 | 9.45 |
| 60 | 10.4 | | 10.2 | 9.05 |
| 75 | 11.0 | | 11.0 | 9.06 |
| 90 | 10.8 | | 10.4 | 9.9 |
| 105 | 11 | | 9.06 | 8.8 |
| 120 | 10.4 | | 8.73 | 8.3 |
| 135 | 10.4 | | 8.55 | 8.8 |
| 150 | 8.65 | | 7.63 | 8.4 |
| 165 | 9.45 | | 8.2 | 7.1 |
| 180 | 9.66 | | 7.36 | 7.95 |

The results of the second glycaemic test profile are plotted against the control in FIG. 13.

When participant T2D1 consumed a premeal drink containing 25 mg of Acarbose, 20 g whey protein and 5 g psyllium husk (see example A1c—made according to the method of the invention) immediately prior to 240 g of white rice:

Participant produced a better glycemic profile when compared to control—
lower post-prandial glucose levels from t=105 minutes to t=180 minutes compared to control.

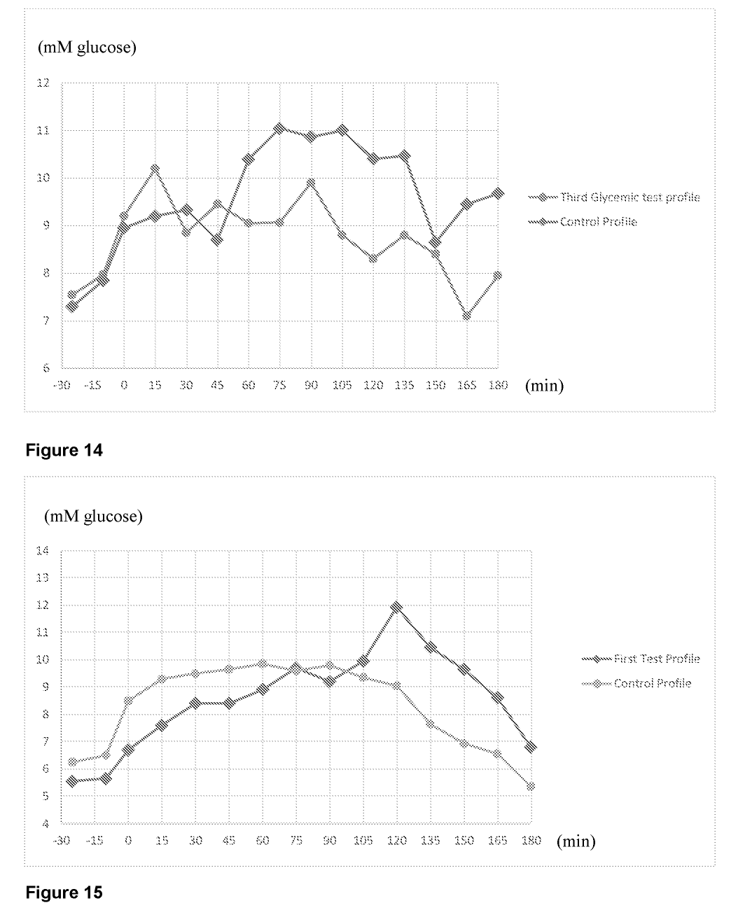

The results of the third glycaemic test profile are plotted against the control in FIG. 14

When participant T2D1 consumed a premeal drink containing 25 mg of Acarbose, 20 whey protein and 5 g guar gum (see example A1a—made according to the method of the invention) immediately prior to 240 g of white rice:

Participant produced a better glycemic profile when compared to control—
lower peak glucose height compared to control (0.85 mmol/L lower than control)
blood glucose stayed lower for longer (from t=60 minutes to t=150 minutes) compared to control

### Example B4

A subject with type 2 diabetes (T2D5, Female, Age 65,) was given a standard white bread breakfast, containing 2 slices of white bread+29 g Jam (example A4) and also the same subject was given a tablet containing 25 mg of acarbose with the first bite of the standard white bread breakfast and was tested for post-prandial glycemic profile—this was glycemic control profile. On another day, the same subject was given a pre-meal drink (not according to the invention), which contained 25 mg acarbose and 25 g whey protein (see example A1d above—this pre-meal drink was consumed 15 minutes before the meal). This was the first glycemic test profile. On another day, the same subject was given a pre-meal drink (not according to the invention), which contained 25 mg acarbose and 12.5 g milk protein isolate and 10.17 g whey protein and 0.325 g guar gum and 0.125 g xanthan gum (see example A1e above—this pre-meal drink was consumed immediately before the meal). This was the second glycemic test profile.

Control—25 mg of acarbose with first mouthful of 2 slices of white bread+29 g Jam (see example A4a)

1^{st} Glycemic Test—25 mg of Acarbose mixed into a premeal drink (not according to the invention) containing 25 g whey protein, (see example A1d) consumed 15 minutes prior to 2 slices of white bread+29 g Jam.

2^{nd} Glycemic Test—25 mg of Acarbose mixed into a premeal drink (not according to the invention) containing 10.17 g whey protein and 12.5 g milk protein isolate and 0.325 g guar gum and 0.125 g xanthan gum, (see example A1e) consumed immediately prior to 2 slices of white bread+29 g Jam.

TABLE 7
Participant T2D5

| | | First | Second |
|---|---|---|---|
| | Glycemic | glycemic | glycemic |
| Time | control | test | test |
| after | profile | profile | profile |
| eating | (mM blood | (mM blood | (mM blood |
| (mins) | glucose) | glucose) | glucose) |
| −25 | 6.25 | 5.55 | 6.93 |
| −10 | 6.5 | 5.65 | 6.9 |
| 0 | 8.5 | 6.7 | 7.56 |
| 15 | 9.3 | 7.6 | 8.33 |
| 30 | 9.5 | 8.4 | 8.37 |
| 45 | 9.65 | 8.4 | 8.35 |
| 60 | 9.85 | 8.9 | 9.1 |
| 75 | 9.6 | 9.7 | 10.2 |
| 90 | 9.8 | 9.2 | 11.0 |
| 105 | 9.35 | 9.95 | 11.5 |
| 120 | 9.05 | 11.9 | 10.4 |
| 135 | 7.65 | 10.4 | 9.45 |
| 150 | 6.93 | 9.65 | 8.4 |
| 165 | 6.55 | 8.6 | 7.2 |
| 180 | 5.35 | 6.8 | 6.85 |

The results of the first test profile are plotted with the control profile in FIG. 15.

When participant T2D5 consumed a premeal drink containing 25 mg of Acarbose and 25 g whey protein, (see example A1d—not according to the invention) 15 minutes prior to consuming 2 slices of white bread+29 g Jam.

Participant produced a worse glycemic profile when compared to control—
T2D5 produced 2.0 mmol higher post prandial peak height at t=120 minutes when compared against control.

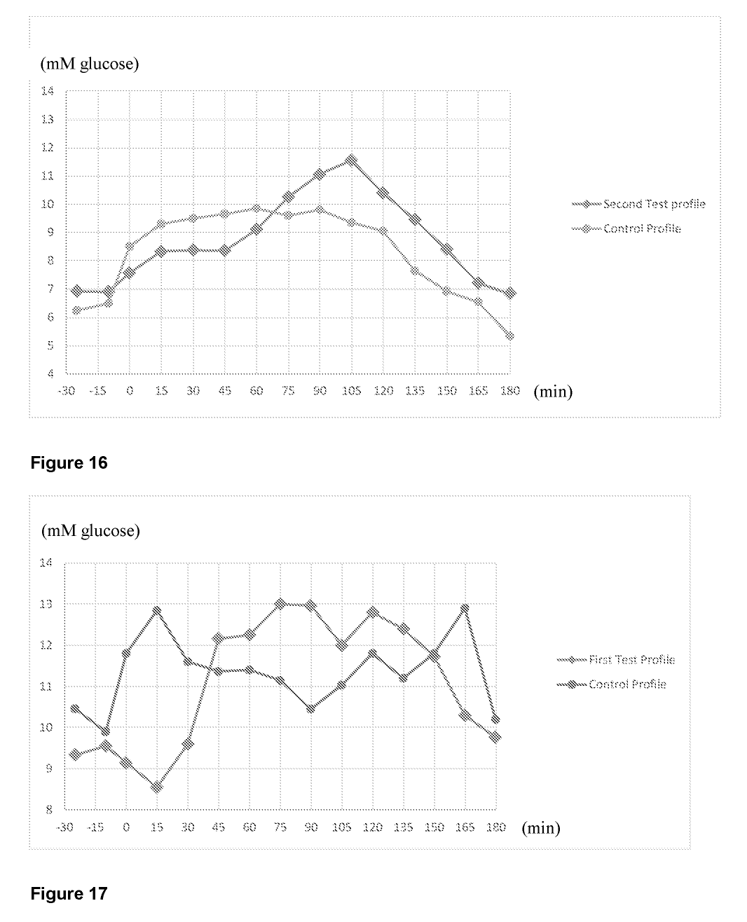

The results of the second test profile are plotted with the control in FIG. 16.

When participant T2D5 consumed a premeal drink containing 25 mg of Acarbose 10.17 g whey protein and 12.5 g milk protein isolate and 0.325 g guar gum and 0.125 g xanthan gum, (see example A1e—not according to the invention) immediately prior to consuming 2 slices of white bread+29 g Jam

Participant produced a worse glycemic profile when compared to control—
T2D5 produced 2.0 mmol higher post prandial peak height at t=105 min compared against control.

### Example B5

A subject with type 2 diabetes (T2D9, Female, Age 61,) was given a standard white bread breakfast, containing 2 slices of white bread+29 g Jam (example A4) and also the same subject was given a tablet containing 25 mg of acarbose with the first bite of the standard white bread breakfast and was tested for post-prandial glycemic profile—this was the control glycemic profile. On another day, the same subject was given a pre-meal drink, which contained 25 mg acarbose and 25 g whey protein (see example A1d above—this pre-meal drink was consumed 15 minutes before the meal). This was the first test glycemic profile. On another day, the same subject was given a pre-meal drink, which contained 25 mg acarbose and 12.5 g milk protein isolate and 10.17 g whey protein and 0.325 g guar gum and 0.125 g xanthan gum (see example A1e above—this pre-meal drink was consumed immediately before the meal). This was the second glycemic test profile.

Control—25 mg of acarbose with first mouthful of 2 slices of white bread+29 g Jam (see example A4a).

1^{st} Glycemic Test—25 mg of Acarbose mixed into a premeal drink (not according to the invention) containing 25 g whey protein, (see example A1d) consumed 15 minutes prior to 2 slices of white bread+29 g Jam.

2^{nd} Glycemic Test—25 mg of Acarbose mixed into a premeal drink (not according to the invention) containing 10.17 g whey protein and 12.5 g milk protein isolate and 0.325 g guar gum and 0.125 g xanthan gum, (see example A1e) consumed immediately prior to 2 slices of white bread+29 g Jam.

TABLE 8

| | | First | Second |
|---|---|---|---|
| | Glycemic | glycemic | glycemic |
| Time | control | control | control |
| after | profile | profile | profile |
| eating | (mM blood | (mM blood | (mM blood |
| (mins) | glucose) | glucose) | glucose) |
| −25 | 10.4 | 9.3 | 8.7 |
| −10 | 9.9 | 9.55 | 8.86 |
| 0 | 11.8 | 9.15 | 10.3 |
| 15 | 12.8 | 8.55 | 10.8 |
| 30 | 11.6 | 9.6 | 12.5 |
| 45 | 11.3 | 12.1 | 13.1 |
| 60 | 11.4 | 12.2 | 13.5 |
| 75 | 11.1 | 13 | 14.1 |
| 90 | 10.4 | 12.9 | 14.2 |
| 105 | 11.0 | 12 | 14.3 |
| 120 | 11.8 | 12.8 | 12.1 |
| 135 | 11.2 | 12.4 | 11.7 |
| 150 | 11.8 | 11.7 | 11.5 |
| 165 | 12.9 | 10.3 | 10.1 |
| 180 | 10.2 | 9.76 | 9.9 |

The results of the first test profile and control are plotted in FIG. 17.

When participant T2D9 consumed a premeal drink containing 25 mg of Acarbose and 25 g whey protein, (see example A1d—not according to the invention) 15 minutes prior to consuming 2 slices of white bread+29 g Jam.

Participant produced a worse glycemic profile when compared to control—
Equivalent peak height compared to control
Blood glucose stayed much higher for longer when compared to control (from t=30 minutes to t=135 minutes)

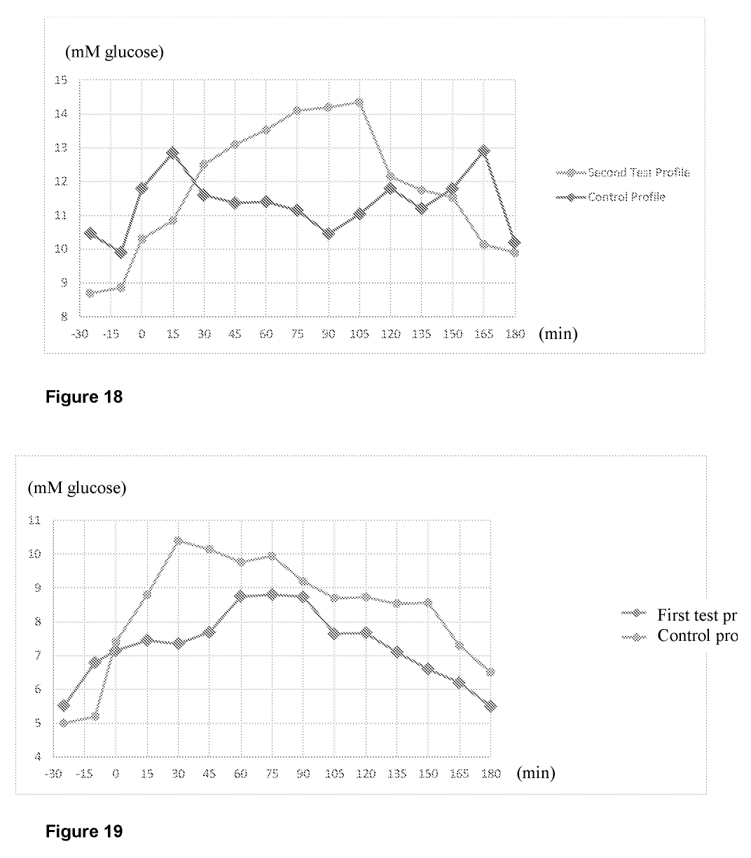

The results of the second test profile and control are plotted in FIG. 18.

When participant T2D9 consumed a premeal drink containing 25 mg of Acarbose 10.17 g whey protein and 12.5 g milk protein isolate and 0.325 g guar gum and 0.125 g xanthan gum, (see example A1e—not according to the invention) immediately prior to consuming 2 slices of white bread+29 g Jam

Participant produced a worse glycemic profile when compared to control—
1.2 mmol higher post prandial peak height at t=105 min
blood glucose stayed higher for longer when compared to control (from t=30 minutes to t=135 minutes) than control

### Example B6

A subject with type 2 diabetes (T2D5, Female, Age 65,) was given a standard white rice breakfast, containing 240 g cooked white rice (example A3a) and also the same subject was given a tablet containing 25 mg of acarbose with the first bite of the standard white bread breakfast and was tested for post-prandial glycemic profile—this was the control glycemic profile. On another day, the same subject was given a pre-meal drink, which contained 25 mg acarbose and 12.5 g milk protein isolate and 10.17 g whey protein and 1.475 g guar gum and 0.125 g xanthan gum (see example A1f above—this pre-meal drink was consumed immediately before the meal). This was the first glycemic test profile.

Control—25 mg of acarbose with first mouthful of 240 g cooked white rice (see example A3a)

1st Glycemic Test—25 mg of Acarbose mixed into a premeal drink (according to the invention) containing 10.17 g whey protein and 12.5 g milk protein isolate and 1.475 g guar gum and 0.125 g xanthan gum, (see example A1f) consumed immediately prior to 240 g cooked white rice.

TABLE 9

| Time after | Glycemic | First glycemic |
|---|---|---|
| eating | control profile | test profile |
| (mins) | (mM blood glucose) | (mM blood glucose) |
| −25 | 5 | 5.5 |
| −10 | 5.2 | 6.8 |
| 0 | 7.4 | 7.15 |
| 15 | 8.8 | 7.45 |
| 30 | 10.4 | 7.35 |
| 45 | 10.15 | 7.7 |
| 60 | 9.76 | 8.75 |
| 75 | 9.95 | 8.8 |
| 90 | 9.2 | 8.75 |
| 105 | 8.7 | 7.65 |
| 120 | 8.73 | 7.66 |
| 135 | 8.53 | 7.1 |
| 150 | 8.56 | 6.6 |
| 165 | 7.3 | 6.2 |
| 180 | 6.5 | 5.5 |

The results of the first test profile and control are plotted in FIG. 19.

When participant T2D5 consumed a premeal drink containing 25 mg of Acarbose 10.17 g whey protein and 12.5 g milk protein isolate and 1.475 g guar gum and 0.125 g xanthan gum, (see example A1f—according to the invention) immediately prior to consuming 240 g Cooked white rice.

Participant produced a better glycemic profile when compared to control—
lower post prandial peak height at t=75 min compared to control
blood glucose stayed lower for longer when compared to control (from t=0 minutes to t=180 minutes)

### Example B7

A subject with type 2 diabetes (T2D1, Female, Age 62,) was given a standard white rice breakfast, containing 240 g cooked white rice (example A3a) and also the same subject was given a tablet containing 25 mg of acarbose with the first bite of the standard white bread breakfast and was tested for post-prandial glycemic profile—this was the control glycemic profile. On another day, the same subject was given a pre-meal drink, which contained 25 mg acarbose and 12.5 g milk protein isolate and 10.17 g whey protein and 1.475 g guar gum and 0.125 g xanthan gum (see example A1f above—this pre-meal drink was consumed immediately before the meal). This was the first glycemic test profile.

Control—25 mg of acarbose with first mouthful of 240 g cooked white rice (see example A3a).

1st Glycemic Test—25 mg of Acarbose mixed into a premeal drink (according to the invention) containing 10.17 g whey protein and 12.5 g milk protein isolate and 1.475 g guar gum and 0.125 g xanthan gum, (see example A1f) consumed immediately prior to 240 g cooked white rice.

TABLE 10

| Time after | Glycemic | First glycemic |
|---|---|---|
| eating | control profile | test profile |
| (mins) | (mM blood glucose) | (mM blood glucose) |
| −25 | 7.3 | 8.1 |
| −10 | 7.85 | 8.7 |
| 0 | 8.95 | 9.26 |
| 15 | 9.2 | 9.46 |
| 30 | 9.32 | 10.2 |
| 45 | 8.7 | 9.03 |
| 60 | 10.4 | 9.7 |
| 75 | 11.0 | 9.75 |
| 90 | 10.8 | 9.73 |
| 105 | 11 | 9.03 |
| 120 | 10.4 | 9.05 |
| 135 | 10.4 | 8 |
| 150 | 8.65 | 8.7 |
| 165 | 9.45 | 8.36 |
| 180 | 9.66 | 8.2 |

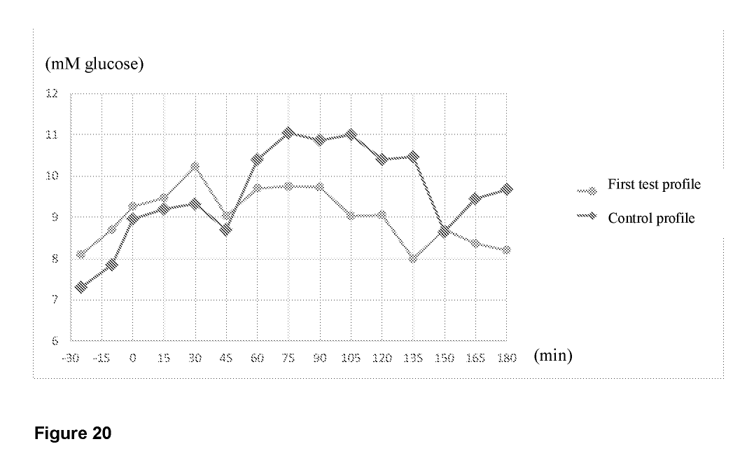

The results of the first test profile and control are plotted in FIG. 20.

When participant T2D1 consumed a premeal drink containing 25 mg of Acarbose 10.17 g whey protein and 12.5 g milk protein isolate and 1.475 g guar gum and 0.125 g xanthan gum, (see example A1f—according to the invention) immediately prior to consuming 240 g Cooked white rice.

Participant produced a better glycemic profile when compared to control—
lower post prandial peak height at t=30 min compared to control
blood glucose stayed lower for longer when compared to control (from t=60 minutes to t=150 minutes) than control.

### Example B8

Formulations were tested by reconstituting ingredients and testing their flow capabilities at 30 seconds after reconstitution.

If after 30 seconds from reconstitution the ingredients cannot be freely poured out of the vessel used to reconstitute them, they would be considered as unsatisfactory drinks.

This involved:

1. mixing ingredients within a ‘shake and take’ container (for 5 seconds)

2. Allow ingredients to rest for 30 seconds

3. Attempt to pour ingredients out of the container.

TABLE 11

| FORMULATION | FLOW AT 30 SECONDS | |
|---|---|---|
| 25 mg Acarbose + 20 g | Pourable liquid, contents | Formulation |
| WPC80 Protein + 5 g | can be emptied from con- | is in accor- |
| guar gum + 150 ml | tainer at t = 30 seconds | dance with |
| water | | invention. |
| 25 mg Acarbose + 12 g | Pourable liquid, contents | Formulation |
| WPC80 Protein + 5 g | can be emptied from con- | is inside of |
| guar gum + 150 ml | tainer at t = 30 seconds | patent claims. |
| water | | |
| 25 mg Acarbose + 12 g | Pourable liquid, contents | Formulation |
| Milk Protein Isolate + | can be emptied from con- | is inside of |
| 7 g psyllium husk + | tainer at t = 30 seconds | patent claims. |
| 150 ml water | | |
| 25 mg Acarbose + 8 g | Pourable liquid, contents | Formulation |
| Milk Protein Isolate + | can be emptied from con- | is inside of |
| 7 g psyllium husk + | tainer at t = 30 seconds | patent claims. |
| 150 ml water | | |
| 25 mg Acarbose + 20 g | Pourable liquid, contents | Formulation |
| Milk Protein Isolate + | can be emptied from con- | is inside of |
| 7 g psyllium husk + | tainer at t = 30 seconds | patent claims. |
| 150 ml water | | |
| 25 mg Acarbose + 5 g | Too viscous, contents could | Formulation |
| Soy Protein + 9 g | not be emptied from con- | is not of the |
| guar gum + 150 ml | tainer at t = 30 seconds | invention |
| water | | |
| 25 mg Acarbose + 7 g | Too viscous, contents could | Formulation |
| Soy Protein + 9 g | not be emptied from con- | is outside of |
| guar gum + 150 ml | tainer at t = 30 seconds | patent claims. |
| water | | |
| 25 mg Acarbose + 5 g | Too viscous, contents could | Formulation |
| MPI + 9 g guar gum + | not be emptied from con- | is outside of |
| 150 ml water | tainer at t = 30 seconds | patent claims. |
| 25 mg Acarbose + 5 g | Too viscous, contents could | Formulation |
| WPC80 + 5 g guar gum + | not be emptied from con- | is outside of |
| 150 ml water | tainer at t = 30 seconds | patent claims. |

### Example B9

Formulations were further tested for shearbanding and eccentric shearbanding:

TABLE 12

| | | Eccentric | |
|---|---|---|---|
| | Shearbanding | Shearbanding | |
| Formulation | Yes/No | Yes/No | |
| 25 mg Acarbose + 20 g | Yes | Yes | Formulation |
| WPC80 Protein + 5 g | | | is in accor- |
| guar gum + 150 ml | | | dance with |
| water | | | invention. |
| 25 mg Acarbose + 12 g | Yes | Yes | Formulation |
| WPC80 Protein + 5 g | | | is in accor- |
| guar gum + 150 ml | | | dance with |
| water | | | invention. |
| 25 mg Acarbose + 12 g | Yes | Yes | Formulation |
| Milk Protein Isolate + | | | is in accor- |
| 7 g psyllium husk + | | | dance with |
| 150 ml water | | | invention. |
| 25 mg Acarbose + 10 g | Yes | Yes | Formulation |
| WPC80 Protein + 2.5 g | | | is in accor- |
| guar gum + 75 ml | | | dance with |
| water | | | invention. |
| 25 mg Acarbose + 6 g | Yes | Yes | Formulation |
| WPC80 Protein + 2.5 g | | | is in accor- |
| guar gum + 75 ml | | | dance with |
| water | | | invention. |
| 25 mg Acarbose + 6 g | Yes | Yes | Formulation |
| Milk Protein Isolate + | | | is in accor- |
| 3.5 g psyllium husk + | | | dance with |
| 75 ml water | | | invention. |
| 25 mg Acarbose + 10.17 | No | No | Formulation |
| gWPC80 + 12.5 g MPI + | | | is not of |
| 0.325 g guar gum + | | | the inven- |
| 0.125 g xanthan gum | | | tion. |
| (combined 0.45 g sol- | | | |
| uble fibre content) + | | | |
| 150 ml water | | | |
| 25 mg Acarbose + 25 | No | No | Formulation |
| gWPC80 + 150 ml water | | | is not of |
| | | | the inven- |
| | | | tion. |
| 25 mg Acarbose + 5 g | No | No | Formulation |
| Milk Protein Isolate + | | | is not of |
| 5 g psyllium husk + | | | the inven- |
| 150 ml water | | | tion. |
| 25 mg Acarbose + 5 | No | No | Formulation |
| gWPC80 + 6.25 g MPI + | | | is not of |
| 0.15 g guar gum + | | | the inven- |
| 0.6 g xanthan gum | | | tion. |
| (combined 0.45 g sol- | | | |
| uble fibre content) + | | | |
| 75 ml water | | | |
| 25 mg Acarbose + 12.5 | No | No | Formulation |
| gWPC80 + 75 ml water | | | is not of |
| | | | the inven- |
| | | | tion. |
| 25 mg Acarbose + 2.5 g | No | No | Formulation |
| Milk Protein Isolate + | | | is not of |
| 2.5 g psyllium husk + | | | the inven- |
| 75 ml water | | | tion. |

1. A powder composition for preparation of a drink for control of post prandial glycaemic profile, the powder composition comprising:
alpha-glucosidase inhibitor selected from the group consisting of acarbose, miglitol, voglibose and mixtures thereof, in an amount in the range of from 0.01% to 15% w/w of the powder composition;
viscosifying agent in an amount in the range of 5% to 30% w/w of the powder composition;
protein in an amount in the range of 40% to 90% w/w of the pharmaceutical powder composition.

2. A powder composition according to claim 1, wherein the alpha-glucosidase inhibitor is present in an amount of 0.05% to 5% w/w of the powder composition; a viscosifying agent which is a polysaccharide in an amount in the range of 10-20% w/w of the powder composition and protein present in an amount of 50% to 80% w/w of the powder composition.

3. A powder composition according to claim 1, wherein the alpha-glucosidase inhibitor is acarbose, the viscosifying agent is a galactomannan gum and the protein is a dairy protein.

4. (canceled)

5. A powder composition according to claim 1 in the form of a unit dose for preparation of a drink for control of post prandial glycaemic profile, the unit dose powder composition comprising:
from 5 mg to 250 mg of alpha-glucosidase inhibitor;
protein in an amount of 8 g to 40 g;
viscosifying agent in an amount of from 1 g to 8 g.

6. A powder composition according to claim 5, wherein the alpha-glucosidase inhibitor is acarbose present in an amount of from 5 mg to 100 mg.

7. A powder composition according to claim 5 dispersed in an aqueous drink of volume in the range of from 30 ml to 300 ml.

8. A powder composition according to claim 7, wherein the volume of aqueous drink is from 75 ml to 200 ml.

9. (canceled)

10. A powder composition according to claim 1 further comprising a silica based flow modifier present in the powder composition in an amount of from 0.5% w/w to 2.0% w/w of the powder composition.

11. An aqueous glucosidase inhibitor composition in the form of a drink comprising a powder composition dispersed in an aqueous liquid in ratio of weight of powder components to volume of aqueous liquid of
10-100 mg alpha-glucosidase inhibitor;
8-30 g protein; and
1.5-8 g soluble fibre;
per 150 ml of aqueous liquid.

12. An aqueous alpha-glucosidase inhibitor composition according to claim 11, wherein the alpha-glucosidase inhibitor is acarbose, the protein is dairy protein and the soluble fiber is a galactomannan gum.

13. An aqueous alpha-glucosidase inhibitor composition according to claim 11, wherein the protein is whey protein and the viscosifying agent is guar gum, fenugreek gum or mixture thereof.

14. An aqueous alpha-glucosidase composition according to claim 11, wherein the composition is formulated to exhibit eccentric shear banding flow characteristics.

15. A method of controlling post-prandial glycaemic profile of a subject, comprising:
providing a unit dose of powder composition comprising:
alpha-glucosidase inhibitor in an amount of from 5 mg to 250 mg;
protein in an amount of 8 g to 40 g;
viscosifying agent in an amount of from 1 g to 8 g;
combining the unit dose of powder with an amount of aqueous liquid in the range of from 75 ml to 200 ml to provide a drink composition; and
the subject consuming the drink composition.

16. A method according to claim 15, wherein the drink is consumed within 30 minutes to 0 minutes (immediately before) before a meal.

17. A method according to claim 15, wherein the drink is consumed within 5 minutes of mixing the powder with an aqueous liquid.

18. A method according to claim 15, wherein the alpha-glucosidase inhibitor is acarbose in an amount of 5 mg to 100 mg, the protein is dairy protein in an amount of from 8 g to 40 g and the viscosifying agent is galactomannan gum in an amount of from 1 g to 8 g.

19. A method according to claim 15, wherein the subject is suffering diabetes or prediabetes.

20. A method according to claim 15, wherein the alpha-glucosidase inhibitor is acarbose, the protein is whey protein and the viscosifying agent is galactomannan gum.

21. A method according to claim 15, wherein the drink exhibits centric-shearbanding.

22.-23. (canceled)

24. A powder composition according to claim 5 in the form of a unit dose, wherein the viscosifying agent is present in an amount sufficient to provide an aqueous mixture of the powder with a viscosity of at least 600 cp within 15 minutes of a unit dose of the powder being mixed with 150 ml water.
